(19) United States
(12) Patent Application Publication
(10) Pub. No.: US 2021/0249106 A1
(43) Pub. Date: Aug. 12, 2021

(54) SYSTEMS MODELING METHOD FOR MOLECULAR BIOLOGY

(71) Applicant: Dmytro Vladimirovich Poluektov, Seattle, WA (US)

(72) Inventor: Dmytro Vladimirovich Poluektov, Seattle, WA (US)

(21) Appl. No.: 16/785,586

(22) Filed: Feb. 8, 2020

(51) Int. Cl. G16B 45/00; G06F 30/12

(52) U.S. Cl. CPC G16B 45/00; G06F 30/12

(57) ABSTRACT

To provide a hierarchical layered hybrid tree chord method for bio-molecular organism functions modeling which enables the display of multiple structural and connective constructs. Composed of tree diagram logical layout in chord diagram clockwise or counterclockwise physical layout with regular polygons legend for nodes. The polygon shapes are used to contain biochemical processes for their respective hierarchical network layers and to interconnect said processes with other layers and other organisms' diagrams. Regular polygon node legend side count indicates bio-molecular layer number where higher complexity bio-molecular layers are represented by higher N sides regular polygons.

### FIELD OF THE INVENTION

The present invention relates to Object Modeling Technique (OMT) design method.

### BACKGROUND OF THE INVENTION

In general, biochemistry, organic chemistry and molecular biology are considered to be complex subjects due to a very large number of bio-molecular components and pathways interacting with each other in varying degrees. To work in the field, generally “Bachelor's and master's degree holders qualify for some entry-level positions in biochemistry and biophysics.” as generally agreed upon and informed by the Bureau of Labor Statistics (BLS, 2020).

Yet biochemistry is not the only field with such complex infrastructure. Telecommunications information pathways are also very complex. This is in large part due to the interplay of the assorted application specific wired and wireless legacy and next generation technologies that all have to inter-operate with each other. Nevertheless, generally Telecommunications field is not necessarily considered intractably complex. Telecommunications programs are common in trade schools and technical two year colleges, since organizations often “may require only a post-secondary certificate or an associate's degree.” (BLS, 2020). Both fields of Biochemistry and Telecommunications therefore have complex systems pathways, but apparently require vastly differing qualifications.

A likely reason for the relatively low industry entry qualifications requirements within Telecommunications field appears to be the unifying, fundamental and simple data communications model—the OSI management framework model that is widely used in the industry. It abstracts the various functional levels of digital and analog communications into functional levels. Its seven layers are used as originally published by the International Standards Organization (ISO, 1989). However such a general purpose modeling method does not appear to exist within the field of molecular biology.

### SUMMARY OF THE INVENTION

The object of the present invention is to provide an OMT design method which can facilitate molecular biology biochemical modeling on the basis of viewing the whole system, including both internal structure and biochemical networks as well as external structures and biochemical networks. The OMT model design method performs this goal by modeling a target system based on integration of hierarchical tree diagrams and chord diagrams. The structure of the model is annotated using regular geometric figure profiles for storing and indicating the hierarchical tree diagram information. More specifically, the internal and external biochemical networks are then linked in the model through the stated regular geometric figure profiles.

According to this OMT design method, an optimized, purpose specific Graphical User Interface (GUI) computer program can then be developed to efficiently model large scale molecular biology systems and link to the underlying biochemical information databases through reference links attached to the models' figure profiles.

### BRIEF DESCRIPTION OF THE DRAWINGS

For a more complete understanding of the present invention and the advantages thereof, reference is now made to the following description taken in conjunction with the accompanying drawings.

FIG. 1 is a tree block diagram showing a general purpose molecular biology hierarchical model.

FIG. 2 is the model's legend diagram showing regular geometric figures to be mapped to represent levels of the molecular biology hierarchical model.

FIG. 3 is a model diagram showing a complete in vivo molecular biology system using the stated geometric figures.

FIG. 4 is a model diagram showing the model's capability to display internal biochemical networks.

FIG. 5 is a model diagram showing the model's capability to display external biochemical networks.

FIG. 6 is a model diagram showing the model's capability to display organism and environmental biochemical interactions.

FIG. 7 is a model diagram showing the model's capability to display internal biochemical structures.

### DETAILED DESCRIPTION OF INVENTION

The present invention provides an OMT design and display method which can facilitate biochemical systems organization in pursuit of optimizing biochemical systems purpose comprehension in education and large scale biochemical systems organization in scientific research.

Although genome interaction chord diagrams can provide maps of genome interactions, such diagrams do not possess functionality for holistically handling molecular biology systems and for treatment of holistic interaction between other such systems and between the environment.

Under such circumstances, a comprehensive modular method for handling molecular biology systems is needed. With such a system, comprehensive addressing of all biochemical components involved can be accomplished. Such a system is presented here, integrating the structural and connective components to be infinitely extensible. This integration is attained through leveraging the synthesis of graph theory's tree diagram models and chord diagram models. The consistent legend of the model also facilitates uniform presentation.

This synthesis is generated through linking both model types through abstractly defined regular geometric shapes. The linking regular geometric shapes are specifically purpose defined to abstractly represent the tree model hierarchical levels. Specifically, the number of regular sides characteristics of regular polygons can be used to represent the layer level numbers of the hierarchical molecular biology model. Thus structure bearing abstract geometric shapes can be integrated as nodes into chord diagrams to be diagrammatically used in biochemical chord network diagrams. The space of the abstract structure bearing regular geometric shape nodes can then also be utilized to directly contain within them specific biochemical structure information.

Hereinafter, an embodiment, which is an OMT design method system, applied to sample biochemical molecular biology systems will be explained based on the accompanying drawings.

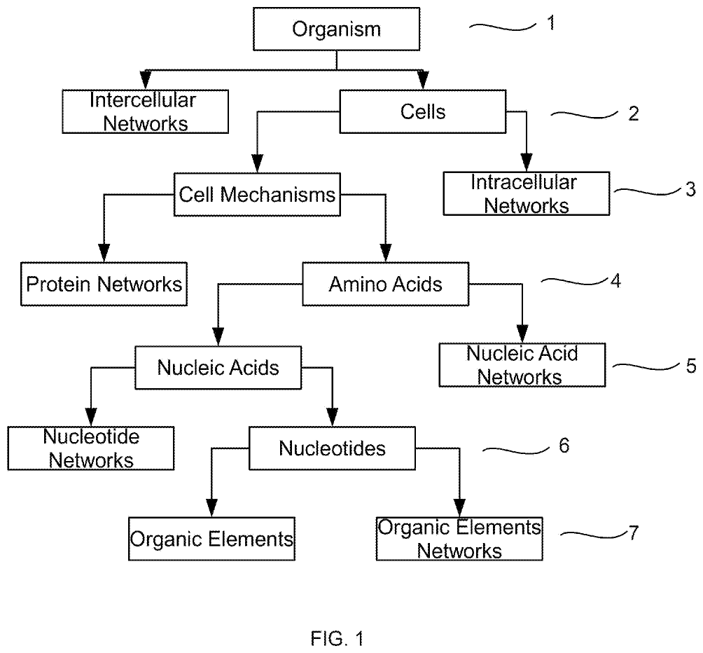

As shown in FIG. 1, the OMT design molecular biology system includes a structured tree summary of biochemical systems. The hierarchical tree model is composed of layer seven—organism (network of cells)—1, layer six—cells (protein networks)—2, layer five—proteins—3, layer four—amino acids—4, layer five—nucleic acids—5, layer six—nucleotides—6, layer one—organic and inorganic chemical elements—7. The basis for this structure is that as per common biochemical knowledge, the functions listed in higher numbered layers are dependent on the functions listed in the lower numbered layers.

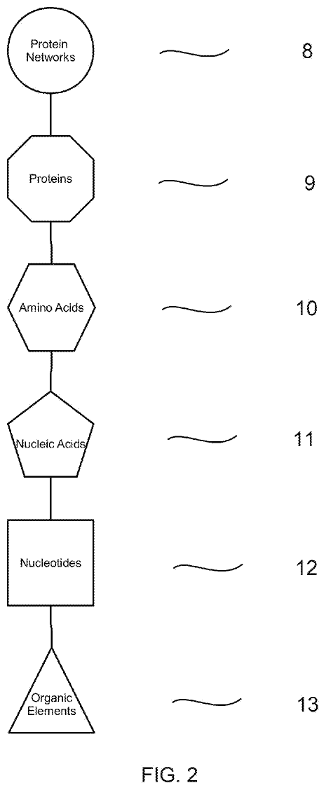

The proposed model's sample legend of regular shapes mapped to the functions of FIG. 1 on the basis of edge counts being mapped to the represented biochemical structures level is shown in FIG. 2. The lowest molecular biology level one of (mostly organic) chemical elements is mapped to a regular polygon of fewest N possible sides, also known as an equilateral triangle 13. The second level of molecular biology—nucleotides—is mapped to the next regular polygon of fewest N possible sides, N+1, also known as a square 12. The third level of molecular biology—nucleic acids—is mapped to the next regular polygon of fewest N possible sides, N+2, also known as a pentagon 11. The forth level of molecular biology—amino acids—is mapped to the next regular polygon of fewest N possible sides, N+3, also known as a hexagon 10. The fifth level of molecular biology—proteins—skips the next N+1 regular polygon known as heptagon and is mapped to the next regular polygon of fewest N possible sides, N+5, also known as a octagon 11. The reason for skipping the heptagon is that the legend picked is designed to be easily readable in large diagrams and heptagons do not appear to be easily readable to due their illusion of appearing asymmetric despite being actually symmetric, similar to nonagons and other odd edge count regular polygons of high edge count. The highest on the cellular level molecular biology level six—protein networks or cells themselves—is mapped to a regular polygon of highest N possible sides or in other words of infinite number of sides, also known as a circle 8.

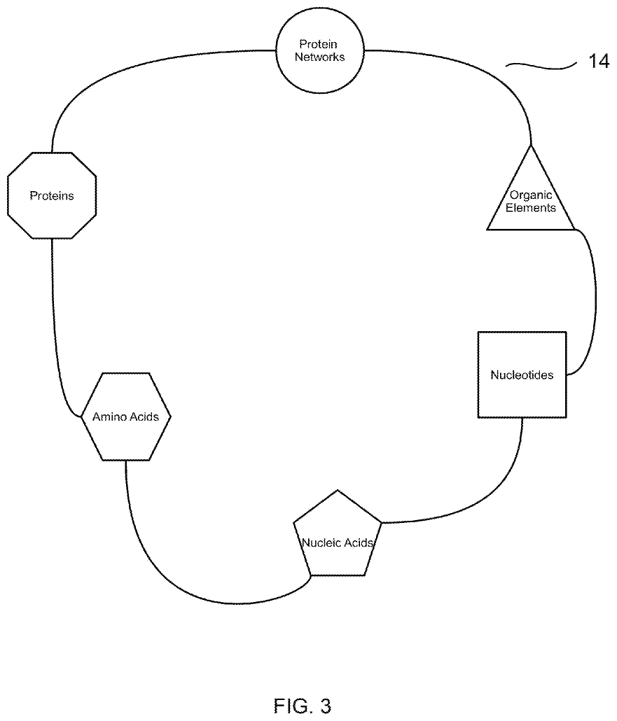

In FIG. 3, the simplified tree model diagram layout constructed in FIG. 2 is taken and is then converted into a chord diagram model. This is accomplished in FIG. 3 by linking the highest and the lowest level model's ends 14. This also makes sense from the biochemical logistical perspective. The highest order layer that is cellular protein interaction networks is composed of proteins from the highest order complexity perspective. However cells and their proteins and protein networks are fundamentally composed from organic elements from the lowest order complexity perspective. Thus the basic hybrid tree-chord diagram cell model is completed.

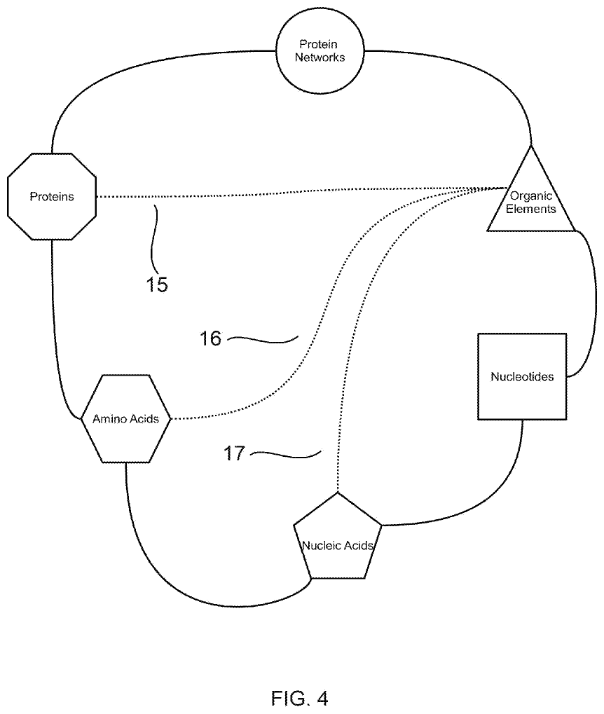

The application of the completed proposed herein molecular biology cellular model to display internal cellular interaction networks is shown in a basic example in FIG. 4. As previously stated, the lowest layer one of organic elements forms both the layer two of nucleotides and the highest layer six of protein networks where they are also composed of organic elements. However all the complexity layers of the cell are composed and built of organic elements and this relationship as a sample is shown here. This includes layer one to layer five 15, layer one to layer four 14 and layer one to layer three 17.

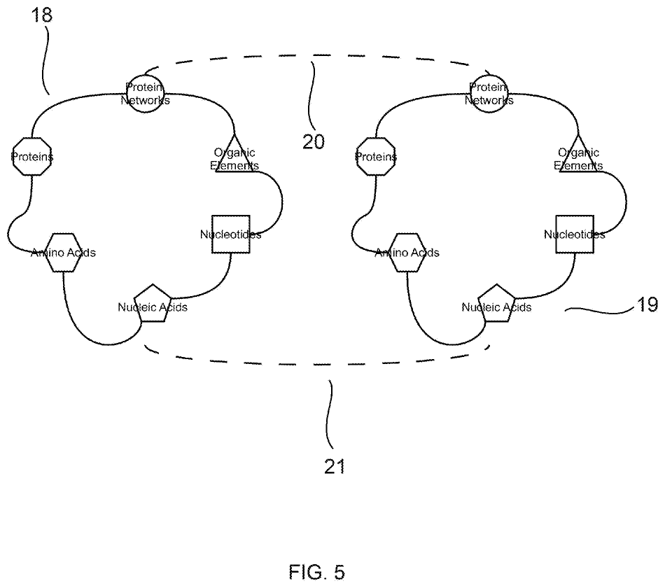

The application of the completed proposed herein molecular biology cellular model to display inter cellular interactions is shown in FIG. 5. This basic example displays two cellular systems (cells) 18, 19, interacting with each other via organelles such as cell membrane on the protein networks layer 20 and exchanging DNA/RNA information on the nucleic acids layer 21.

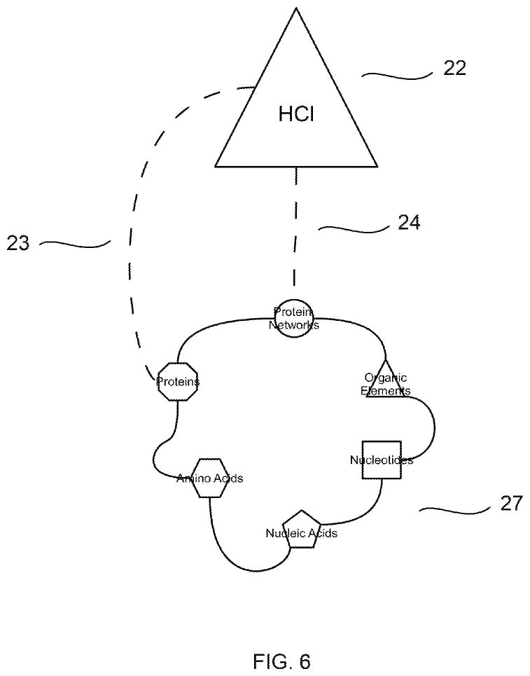

The herein proposed model shows cellular interaction with the environment in FIG. 6. A cell 27 is shown interacting with Hydrogen Chloride acid HCl represented in the chemical elements layer by the equilateral triangle 22. The acid is shown interacting with (dissolving) the proteins 23 and protein networks 24 of the cell walls of the cell.

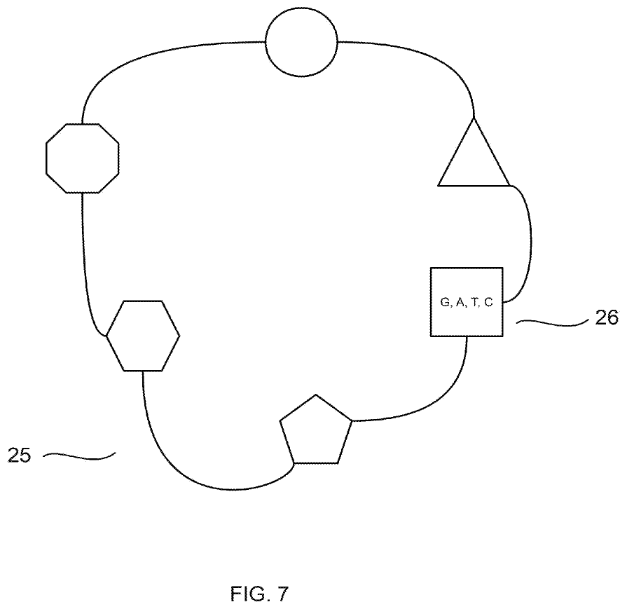

Finally, the ability of the herein model to represent internal cellular biochemical functions is shown in FIG. 7. In the given example of the cell 25, the representative nucleotide references are shown 26.

It is noted that the foregoing has outlined some of the more pertinent objects and embodiments of the present invention. This invention may be used for many applications. Thus, although the description is made for particular arrangements and methods, the intent and concept of the invention is Suit able and applicable to other arrangements and applications. It will be clear to those skilled in the art that modifications to the disclosed embodiments can be effected without departing from the spirit and scope of the invention. The described embodiments ought to be construed to be merely illustrative of Some of the more prominent features and applications of the invention. Other beneficial results can be realized by applying the disclosed invention in a different manner or modifying the invention in ways known to those familiar with the art.

What is claimed is:

1. An OMT tree diagram hierarchical layered design of molecular biology model.

2. Chord diagram layout format encoding the tree diagram to build the hybrid tree-chord molecular biology logical diagram model.

3. The format of the stated chord diagram utilizing regular geometric shapes and their geometric qualities for connectivity nodes to:
a) visually indicate the stated hierarchical layered design biochemical design and
b) to enable integration of internal biochemical networks and
c) to enable integration of external biochemical networks
d) display specific biochemical structures displayed and contained within the stated nodes that actually form the biochemical networks.
